(19) United States
(12) Patent Application Publication
(10) Pub. No.: US 2003/0004448 A1
(43) Pub. Date: Jan. 2, 2003

(54) Breast bounce inhibitor

(76) Inventor: Carla A. Gregory, Winter Springs, FL (US)

Correspondence Address: EDWARD M. LIVINGSTON, ESQUIRE, 628 Ellen Drive, Post Office Box 1599, Winter Park, FL, 32790, US

(21) Appl. No.: 09/882,944

(22) Filed: Jun. 15, 2001

(51) Int. Cl. A61F 5/00

(52) U.S. Cl. 602/19

(57) ABSTRACT

A breast-bounce inhibitor has a bounce-prevention strap (1) articulated for suspension laterally over tops of a woman's breasts (2). It has bounce-resistant resilience in directions of back-strap attachments (3, 4). The bounce-resistant resilience can be adjustable and the bounce-prevention strap can be joinable intermediate the breasts. A back strap can include a conventional bra back strap (14). The bounce-prevention strap can be integrated into a bra (15).

### BACKGROUND OF THE INVENTION

This invention relates to athletic bras and in particular to bras that prevent discomfort, pain and injury of some women from breast bounce.

Sports bras are well known and highly developed. However, like conventional brassieres, none prevent breast bounce adequately. This lack of adequate bounce prevention appears to result from primary concern about aesthetic breast adornment without due regard for bounce prevention caused by vigorous running, jumping, exercising, playing games, dancing, physical work, fighting and other dynamic activities.

No known prior art describes or teaches a breast-top barrier to upward movement for preventing breast bounce in a manner taught by this invention.

Examples of most-closely related known but different bras are described in the following patent documents:

| U.S. Pat. No. | Inventor | Issue Date |
|---|---|---|
| 5,098,331 | Corrado | 03/24/1992 |
| 5,119,511 | Packer, et al. | 06/09/1992 |
| 4,781,651 | Ekins | 11/01/1988 |
| 1,923,821 | Gudeman | 08/22/1933 |
| 3,189,028 | Dormire | 06/15/1965 |
| 2,662,522 | Muller | 12/15/1953 |
| 5,527,270 | Chase, et al. | 06/18/1996 |
| 5,152,741 | Farnio | 10/06/1992 |
| 4,289,137 | Dell, et al. | 09/15/1981 |
| 4,816,005 | Braaten | 03/28/1989 |

The Corrado patent describes a chest dressing with a flexible bandage-material band on breasts of post-operative breast-surgery patients, but comprehensively limited it from having structure, elasticity or resilience pressure that would impede any breast bounce whatsoever. A single breast movement would distort the bandage-material flexibility. It provides only sedentary breast protection that is completely opposite from breast protection from dynamic body action.

Like most shoulder-strap athletic bras, the cited Ekins patent has breast-top restriction with shoulder straps. This leaves a bounce gap between a strap and base portions of breasts, which requires cup fitting and tie-down that are further different. Fundamentally, bras with shoulder straps mostly press inwardly without restricting upwardly bouncing movement.

The above and other objects, features and advantages of the present invention should become even more readily apparent to those skilled in the art upon a reading of the following detailed description in conjunction with the drawings wherein there is shown and described illustrative embodiments of the invention.

### SUMMARY OF THE INVENTION

Objects of patentable novelty and utility taught by this invention are to provide a breast-bounce inhibitor which:

prevents upward movement of breasts for preventing breast bounce from vigorous body activity;

can be worn for separate bounce prevention in combination with conventional athletic or non-athletic bras;

can be built integrally into an athletic bra for two-way bounce prevention; and

can have resilience-pressure adjustment independently.

This invention accomplishes these and other objectives with a breast-bounce inhibitor having a bounce-prevention strap articulated for suspension laterally over tops of a woman's breasts and having bounce-resistant resilience intermediate back-strap attachments. The bounce-resistant resilience can be adjustable and the bounce-prevention strap can be joinable intermediate the breasts. A back strap can include a conventional bra strap. Bottom bounce-prevention structure with coacting bounce prevention can be included.

### BRIEF DESCRIPTION OF DRAWINGS

This invention is described by appended claims in relation to description of a preferred embodiment with reference to the following drawings which are explained briefly as follows:

FIG. 1 is a side view showing positioning of a bounce-prevention strap separately on a woman's breasts;

FIG. 2 is a front view showing positioning of the bounce-prevention strap separately on a woman's breasts;

FIG. 3 is a perspective view of the bounce-prevention strap;

FIG. 4 is a front view of the bounce-prevention strap with a buckle and resilience adjustors for front fastening and resilience-pressure adjustment;

FIG. 5 is a front view of the bounce-prevention strap with a zipper and resilience adjustors for front fastening and resilience-pressure adjustment;

FIG. 6 is a front view of the bounce-prevention strap with a fastener that includes a fold-over fastener and resilience adjustors for front fastening and resilience-pressure adjustment;

FIG. 7 is a front view of the bounce-prevention strap with fastener materials that include “Velcro” and resilience adjustors for front fastening and resilience-pressure adjustment;

FIG. 8 is a front view of the bounce-prevention strap without front adjustment or fastening for use with back-tie or with pre-stressed resilience;

FIG. 9 is a top view of the bounce-prevention strap on an outside of a bra to which it can be attached or built into;

FIG. 10 is a top view of the bounce-prevention strap on an inside of a bra to which it can be attached or built into;

FIG. 11 is an enlargement view of a right side of the FIG. 9 illustration to show outside attachment to a bra back strap;

FIG. 12 is a partially cutaway enlargement view of a right side of the FIG. 10 illustration to show inside attachment to a bra back strap;

FIG. 13 is a view of a fragmentary portion of the bounce-prevention strap on an outside of bra material on a breast; and

FIG. 14 is a view of a fragmentary portion of the bounce-prevention strap on an inside of bra material on the breast.

### DESCRIPTION OF PREFERRED EMBODIMENT

Listed numerically below with reference to the drawings are terms used to describe features of this invention. These terms and numbers assigned to them designate the same features throughout this description.

| | |
|---|---|
| 1. Bounce-prevention strap | 9. Zippered-length adjusters |
| 2. Breasts | 10. Fastener |
| 3. First back-strap attachment | 11. Fastened-length adjusters |
| 4. Second back-strap attachment | 12. Fastener materials |
| 5. Back strap | 13. Material-end adjusters |
| 6. Buckle | 14. Bra back strap |
| 7. Buckled-length adjusters | 15. Bra |
| 8. Zipper | 16. Bra material |

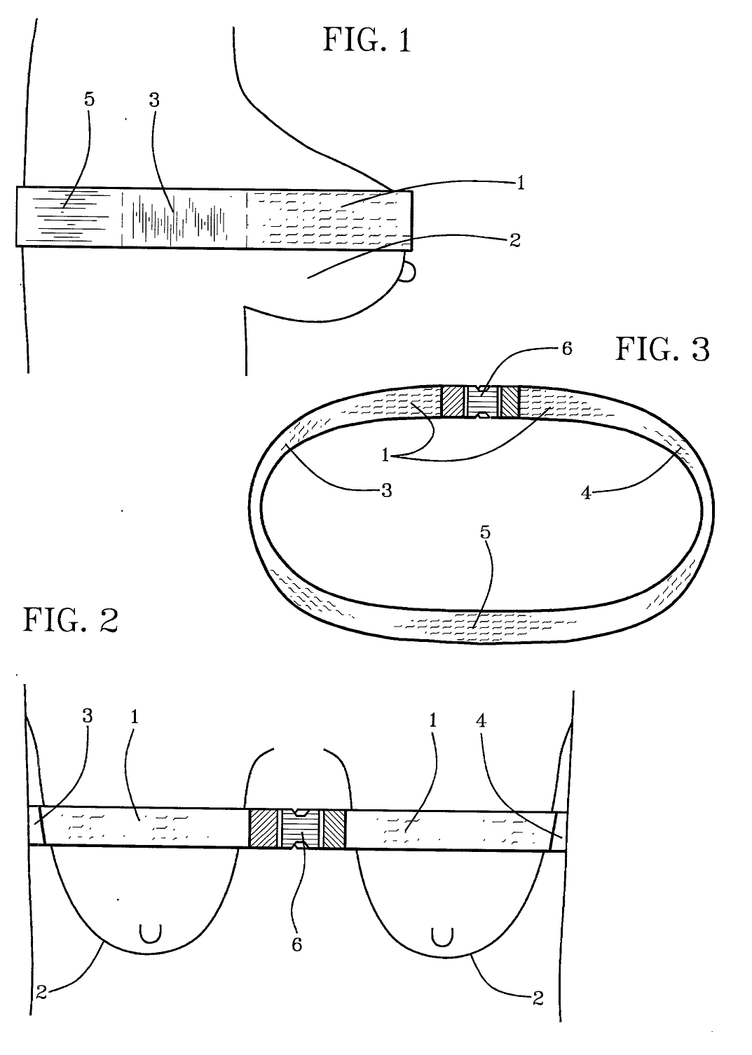

Referring to FIGS. 1-3, a breast-bounce inhibitor has a bounce-prevention strap 1 that is articulated for suspension laterally over tops of a woman's breasts 2. A first back-strap attachment 3 on a first end of the bounce-prevention strap 1 and a second back-strap attachment 4 on a second end of the bounce-prevention strap 1 are attachable to a back strap 5 which can be a bra back strap of a bra. The bounce-prevention strap 1 has bounce-resistance resilience intermediate the first back-strap attachment 3 and the second back-strap attachment 4.

Referring to FIGS. 2-8, articulation for suspension laterally over tops of a woman's breasts 2 can include variable-direction resilience and shaping in addition to straight-line strap resilience that is shown for two-dimensional illustration. Consequently, the bounce-prevention strap 1 can have contoured shapes and resilience intermediate the breasts 2 and the back strap 5. Optionally also with intended contour and resilience, the bounce-prevention strap 1 can be made to back-tie with a buckle 6 or other fastener as shown in FIG. 3 or to front-tie between breasts 2 with the buckle 6 or other fastener as shown in FIGS. 2 and 4-8. Some embodiments can be simpler for short-time and convenience use in comparison to other embodiments that are contoured, sized and shaped for particular breast configurations and sizes.

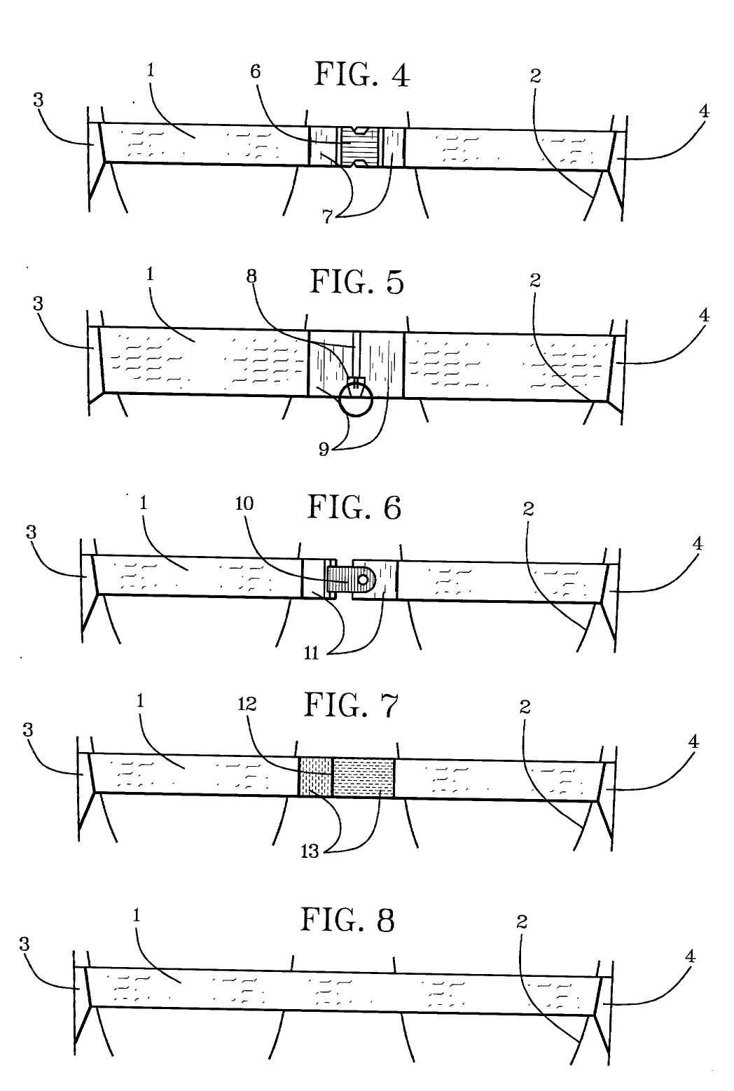

Front-tie, preferably between the breasts 2 can include the buckle 6 with buckled-length adjusters 7 for adjustment of resilience as shown in FIG. 4. Shown in FIG. 5 can be a zipper 8 for wide bounce-prevention straps 1 having zippered-length adjusters 9 for adjustment of resilience. Shown in FIG. 6 can be a fastener 10 having fastened-length adjusters 11 for adjustment of resilience. Shown in FIG. 7 can be a fastener materials 12, including “Velcro”, having material-end adjusters 13 for adjustment of resilience. Optionally as shown in FIG. 8, the bounce-prevention strap 1 can be pre-sized or back-tied not to have front-tie.

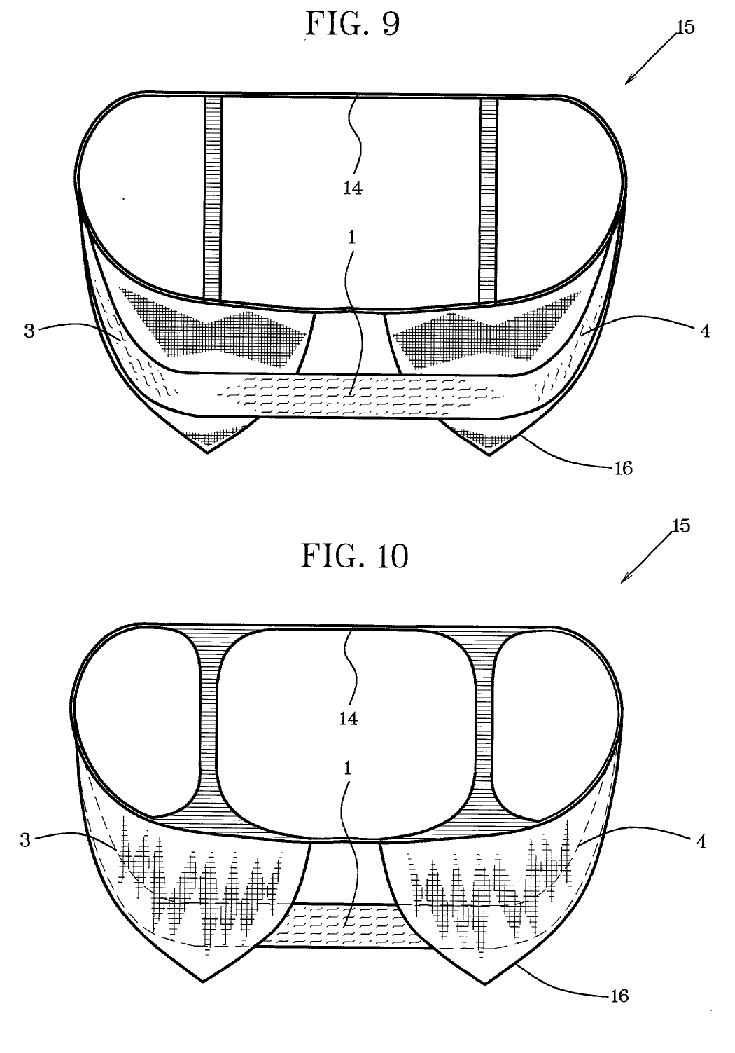

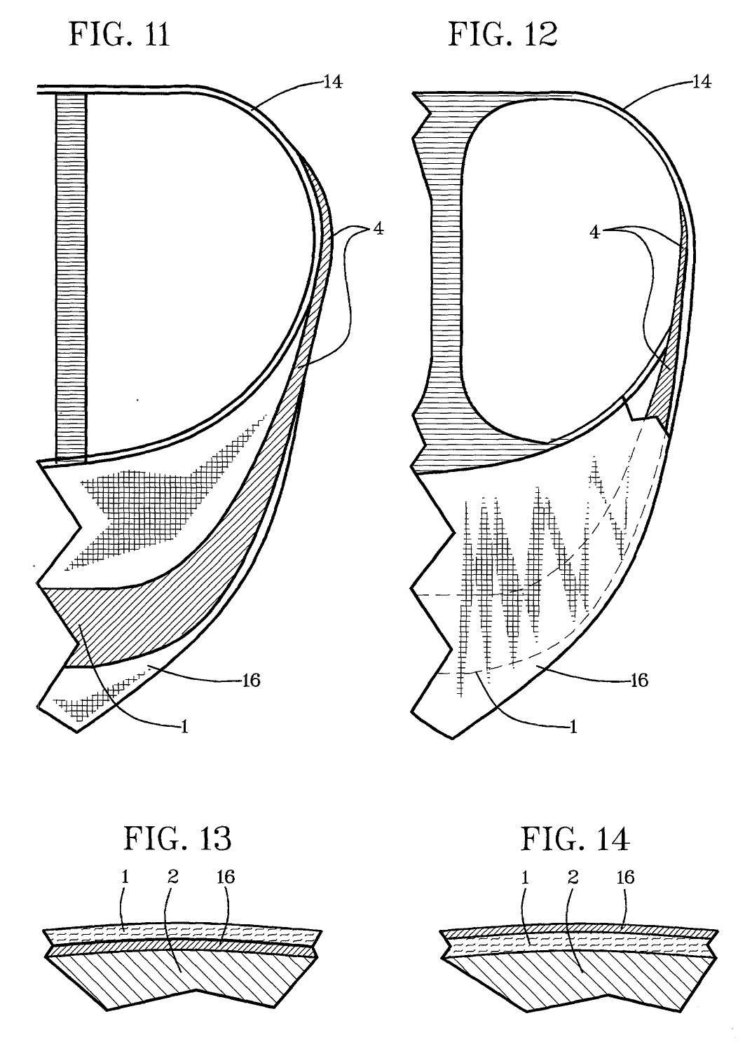

Optionally as described in relation to FIGS. 9-14, the first back-strap attachment 3 can be affixed to or attached removably to a bra back strap 14 of a bra 15. Oppositely disposed correspondingly, the second back-strap attachment 4 can be affixed to or attached removably to the bra back strap 14 of the bra 15.

Further optionally as described in relation to FIGS. 9, 11 and 13, the first back-strap attachment 3 can be affixed to or attached removably to an outside periphery of the first side of the bra back strap 14 for outside positioning on the bra 15. Oppositely disposed correspondingly, the second back-strap attachment 4 can be affixed to or attached removably to the outside periphery of the bra back strap 14 for outside positioning on the bra 15. For the outside positioning, bra material 16 is positioned between the breast 2 and the bounce-prevention strap 1.

Additionally optional as described in relation to FIGS. 10, 12 and 14, the first back-strap attachment 3 can be affixed to or attached removably to an inside periphery of the first side of the bra back strap 14 for inside positioning under the bra 15. Oppositely disposed correspondingly, the second back-strap attachment 4 can be affixed to or attached removably to the inside periphery of the bra back strap 14 for inside positioning under the bra 15. For the inside positioning, the bounce-prevention strap 1 is positioned between the breast 2 and bra material 16.

For integration of the breast-bounce inhibitor into the bra 15, the first back-strap attachment 3 can be a bra-strap extension on a first side of the bra back strap 14 and the second back-strap attachment 4 can be a bra-strap extension on a second side of the bra back strap 14.

A new and useful breast-bounce inhibitor having been described, all such foreseeable modifications, adaptations, substitutions of equivalents, mathematical possibilities of combinations of parts, pluralities of parts, applications and forms thereof as described by the following claims and not precluded by prior art are included in this invention.

What is claimed is:

1. A breast-bounce inhibitor comprising:
a bounce-prevention strap articulated for suspension laterally over tops of a woman's breasts;
a first back-strap attachment on a first end of the bounce-prevention strap;
a second back-strap attachment on a second end of the bounce-prevention strap; and
the bounce-prevention strap having bounce-resistant resilience intermediate the first back-strap attachment and the second back-strap attachment.

2. The breast-bounce inhibitor of claim 1 wherein:
the bounce-restraint resilience is adjustable.

3. The breast-bounce inhibitor of claim 1 wherein:
the bounce-prevention strap includes a length adjuster intermediate the first back-strap attachment and the second back-strap attachment.

4. The breast-bounce inhibitor of claim 1 wherein:
the bounce-prevention strap includes a buckle with which the bounce-prevention strap is joinable intermediate the first back-strap attachment and the second back-s trap attachment.

5. The breast-bounce inhibitor of claim 1 wherein:
the bounce-prevention strap includes a zipper with which the bounce-prevention strap is joinable intermediate the first back-strap attachment and the second back-strap attachment.

6. The breast-bounce inhibitor of claim 1 wherein:
the bounce-prevention strap includes a fastener with which the bounce-prevention strap is joinable intermediate the first back-strap attachment and the second back-strap attachment.

7. The breast-bounce inhibitor of claim 4 and further comprising:
a first length adjuster intermediate the buckle and the first back-strap fastener; and
a second length adjuster intermediate the buckle and the second back-strap fastener.

8. The breast-bounce inhibitor of claim 6 wherein:
the fastener includes an overlap fastener having at least one first fastener member on a first portion of the bounce-prevention strap and at least one oppositely disposed fastener member on a second portion of the bounce-prevention strap.

9. The breast-bounce inhibitor of claim 8 wherein:
the first fastener member includes a first fastener material;
the second fastener member includes a second fastener material; and
the bounce-prevention strap has length adjustment by engaging select lengths of the first fastener material with select lengths of the second fastener material.

10. The breast-bounce inhibitor of claim 9 wherein:
the first fastener material includes a first piece of hook and loop fastening material; and
the second fastener material includes a second piece of hook and loop fastening material which fastens to the first piece of hook and loop fastening material.

11. The breast-bounce inhibitor of claim 1 wherein:
the first back-strap attachment is attachable removably to an outside periphery of a first side of a bra back strap of a bra;
the second back-strap attachment is attachable removably to an outside periphery of a second side of a bra back strap of the bra; and
the bounce-prevention strap is positioned on outside peripheries of bra material.

12. The breast-bounce inhibitor of claim 1 wherein:
the first back-strap attachment is attachable removably to an inside periphery of a first side of a bra back strap of a bra;
the second back-strap attachment is attachable removably to an inside periphery of a second side of the bra back strap of the bra; and
the bounce-prevention strap is positioned intermediate top surfaces of the breasts and inside peripheries of the bra material.

13. A breast-bounce inhibitor comprising:
a bounce-prevention strap articulated for suspension laterally over tops of a woman's breasts;
a first back-strap attachment on a first end of the bounce-prevention strap;
a second back-strap attachment on a second end of the bounce-prevention strap;
the first back-strap attachment is a bra-strap extension from a first side of a bra back strap of a bra;
the second back-strap attachment is a bra-strap extension from a second side of the bra back strap of the bra; and
the bounce-prevention strap having bounce-resistant resilience intermediate the first back-strap attachment and the second back-strap attachment.

14. The breast-bounce inhibitor of claim 13 wherein:
the bounce-restraint resilience is adjustable.

15. The breast-bounce inhibitor of claim 13 wherein:
the bounce-prevention strap includes a length adjuster intermediate the first back-strap attachment and the second back-strap attachment.

16. The breast-bounce inhibitor of claim 13 wherein:
the bounce-prevention strap includes a buckle with which the bounce-prevention strap is joinable intermediate the first back-strap attachment and the second back-strap attachment.

17. The breast-bounce inhibitor of claim 13 wherein:
the bounce-prevention strap includes a zipper with which the bounce-prevention strap is joinable intermediate the first back-strap attachment and the second back-strap attachment.

18. The breast-bounce inhibitor of claim 13 wherein:
the bounce-prevention strap includes a fastener with which the bounce-prevention strap is joinable intermediate the first back-strap attachment and the second back-strap attachment.

19. The breast-bounce inhibitor of claim 16 and further comprising:
a first length adjuster intermediate the buckle and the first back-strap fastener; and
a second length adjuster intermediate the buckle and the second back-strap fastener.

20. The breast-bounce inhibitor of claim 18 wherein:
the fastener includes an overlap fastener having at least one first fastener member on a first portion of the bounce-prevention strap and at least one oppositely disposed fastener member on a second portion of the bounce-prevention strap.

21. The breast-bounce inhibitor of claim 20 wherein:
the first fastener member includes a first fastener material;
the second fastener member includes a second fastener material; and
the bounce-prevention strap has length adjustment by engaging select lengths of the first fastener material with select lengths of the second fastener material.

22. The breast-bounce inhibitor of claim 21 wherein:
the first fastener material includes a first piece of hook and loop fastening material; and
the second fastener material includes a second piece of hook and loop fastening material which fastens to the first piece of hook and loop fastening material.

23. The breast-bounce inhibitor of claim 13 wherein:
the first back-strap attachment is attachable removably to an inside periphery of a first side of a bra back strap of the bra;
the second back-strap attachment is attachable removably to an inside periphery of a second side of the bra back strap of the bra; and
the bounce-prevention strap is positioned intermediate top surfaces of the breasts and inside peripheries of the bra material.

24. The breast-bounce inhibitor of claim 13 wherein:
the first back-strap attachment is attachable removably to an outside periphery of a first side of the bra back strap of the bra;
the second back-strap attachment is attachable removably to an outside periphery of a second side of the bra back strap of the bra; and
the bounce-prevention strap is positioned on outside peripheries of the bra material.
